(19) United States
(12) Patent Application Publication
(10) Pub. No.: US 2003/0135871 A1
(43) Pub. Date: Jul. 17, 2003

(54) Modified railroad worm red luciferase coding sequences

(76) Inventors: Kevin A. Nawotka, Alameda, CA (US); Weisheng Zhang, Fremont, CA (US)

Correspondence Address: COOLEY GODWARD LLP (R&P), FIVE PALO ALTO SQUARE, 3000 EL CAMINO REAL, PALO ALTO, CA, 94306-0663, US

(21) Appl. No.: 10/223,072

(22) Filed: Aug. 15, 2002

Related U.S. Application Data
(60) Provisional application No. 60/312,697, filed on Aug. 15, 2001.
(60) Provisional application No. 60/312,687, filed on Aug. 15, 2001.

(51) Int. Cl. A01K 67/033; C07H 21/04; C12N 5/06

(52) U.S. Cl. 800/8; 435/325; 435/320.1; 536/23.2

(57) ABSTRACT

Native and modified forms of Phrixothrix hirtus red luciferase are described. These native and modified forms of luciferase can be used, for example, as reporter molecules in host cells and/or transgenic animals.

### CROSS-REFERENCE TO RELATED APPLICATIONS

This application is related to U.S. Provisional Patent Applications Serial Nos. 60/312,697, filed Aug. 15, 2001, and 60/312,687, filed Aug. 15, 2001, from which priority is claimed under 35 USC § 119(e)(1), and which applications are incorporated herein by reference in their entireties.

### TECHNICAL FIELD

This invention is in the field of molecular biology and medicine. More specifically, it relates to modified forms of Phrixothrix hirtus (railroad worm) red luciferase. The modified forms of this red luciferase described herein are useful in a wide variety of applications. The present invention describes polynucleotide sequences, polypeptide sequences, expression cassettes, vectors, transformed cells, transgenic animals, and methods of use thereof.

### BACKGROUND

In certain organisms, bioluminescence (the ability to emit light) is mediated by the luciferase enzyme. Photoproteins such as luciferase have been used for more than a decade as biological labels to aid in the study of gene expression in cell culture or using excised tissues (Campbell, A. K. 1988. Chemiluminescence. Principles and applications in biology and medicine. Ellis Horwood Ltd. and VCH Verlagsgesellschaft mbH, Chichester, England; Hastings, J. W. (1996) Gene. 173:5-11; Morrey, J. D., et al., (1992) J. Acquir. Immune Defic. Syndr. 5: 1195-203; Morrey, J. D., et al., (1991) J Viol. 65: 5045-51.). Further, low-light imaging of internal bioluminescent signals has been used to study temporal and spatial gene regulation in relatively thin or nearly transparent organisms (Millar A. J., et al., (1992) Plant Cell 4:1075-87; Stanewsky, R., et al., (1997) EMBO J. 16:5006-18; Brandes C, et al., (1996) Neuron 16:687-92). External detection of internal light penetrating the opaque animal tissues has been described (Contag, P. R., et al., (1998) Nature Med. 4:245-7; Contag, C. H., et al., (1997) Photochem Photobiol. 66:523-31; Contag, C. H., et al., (1995) Mol Microbiol. 18:593-603).

Wild-type and modified luciferase coding sequences have been obtained from lux genes (prokaryotic genes encoding a luciferase activity) and luc genes (eukaryotic genes encoding a luciferase activity), including, but not limited to, the following: B. A. Sherf and K. V. Wood, U.S. Pat. No. 5,670,356, issued Sep. 23, 1997; Kazami, J., et al., U.S. Pat. No. 5,604,123, issued Feb. 18, 1997; S. Zenno, et al, U.S. Pat. No. 5,618,722; K. V. Wood, U.S. Pat. No. 5,650,289, issued Jul. 22, 1997; K. V. Wood, U.S. Pat. No. 5,641,641, issued Jun. 24, 1997; N. Kajiyama and E. Nakano, U.S. Pat. No. 5,229,285, issued Jul. 20, 1993; M. J. Cormier and W. W. Lorenz, U.S. Pat. No. 5,292,658, issued Mar. 8, 1994; M. J. Cormier and W. W. Lorenz, U.S. Pat. No. 5,418,155, issued May 23, 1995; de Wet, J. R., et al, Molec. Cell. Biol. 7:725-737, 1987; Tatsumi, H. N., et al, Biochim. Biophys. Acta 1131:161-165, 1992; and Wood, K. V., et al, Science 244:700-702, 1989; all herein incorporated by reference. Eukaryotic luciferase catalyzes a reaction using luciferin as a luminescent substrate to produce light, whereas prokaryotic luciferase catalyzes a reaction using an aldehyde as a luminescent substrate to produce light. A yellow-green luciferase with an emission peak of about 540 nm is commercially available from Promega, Madison, Wis. under the name pGL3. A red luciferase with an emission peak of about 610 nm is described, for example, in Contag et al. (1998) Nat. Med. 4:245-247 and Kajiyama et al. (1991) Prot. Eng. 4:691-693.

However, prior the present disclosure optimized luciferase sequences obtained from Phrixothrix hirtus (railroad worm or RR) have not been described. Thus, the present invention provides novel luciferase sequences useful in molecular biological studies and methods and for the generation of light-producing transgenic animals.

### SUMMARY OF THE INVENTION

The present invention is directed to sequences encoding functional (e.g., able to mediate the production of light in the presence of an appropriate substrate, for example, luciferin, under appropriate conditions) red luciferase of Phrixothrix hirtus. In one aspect, the invention comprises an isolated polynucleotide having at least about 85% sequence identity to the nucleotide sequence shown in FIG. 1 (SEQ ID NO: 1) or fragments thereof. Preferably, the polynucleotide exhibits at least about 90% identity, more preferably 95% identity, and most preferably 98% identity to the nucleotide sequence shown in FIG. 1 (SEQ ID NO: 1). In certain embodiments, the isolated polynucleotide comprises a polynucleotide consisting of full-length SEQ ID NO: 1. In other embodiments, the sequences of the present invention can include fragments of FIG. 1 (SEQ ID NO: 1), for example, from about 15 nucleotides up to the number of nucleotides present in the full-length sequences described herein (e.g., see the Sequence Listing and Figures), including all integer values falling within the above-described range. For example, fragments of the polynucleotide sequences of the present invention may be 30-60 nucleotides, 60-120 nucleotides, 120-240 nucleotides, 240-480 nucleotides, 480-1000 nucleotides, 1000 to 1641 nucleotides, and all integer values therebetween. In one embodiment, the invention includes a polynucleotide sequence encoding a functional luciferase (i.e., one that is capable of mediating the production of light in the presence of the appropriate substrate under appropriate conditions), wherein the polynucleotide sequence comprises a fragment derived from SEQ ID NO: 1. Further, this aspect of the invention includes modifications of the polynucleotide sequence including, but not limited to, the following: codon optimization for expression in a selected cell type or organism (e.g., mice, Candida, or Cryptococcus); removal/modification of unwanted restriction sites; removal/modification of possible glycosylation sites; removal/modification of C-terminal peroxisome targeting sequences; removal/modification of transcription factor binding sites; removal/modification of palindromes; and/or removal/modification of RNA folding structures.

In another aspect, the invention comprises an isolated polynucleotide having at least about 85% sequence identity to the nucleotide sequence shown in FIG. 3 (SEQ ID NO: 3) or fragments thereof. Preferably, the polynucleotide exhibits at least about 90% identity, more preferably 95% identity, and most preferably 98% identity to the nucleotide sequence shown in FIG. 3 (SEQ ID NO: 3). In certain embodiments, the isolated polynucleotide comprises a polynucleotide consisting of full-length SEQ ID NO: 3. In other embodiments, the sequences of the present invention can include fragments of FIG. 3 (SEQ ID NO: 3), for example, from about 15 nucleotides up to the number of nucleotides present in the full-length sequences described herein (e.g., see the Sequence Listing and Figures), including all integer values falling within the above-described range. For example, fragments of the polynucleotide sequences of the present invention may be 30-60 nucleotides, 60-120 nucleotides, 120-240 nucleotides, 240-480 nucleotides, 480-1000 nucleotides, 1000 to 1641 nucleotides, and all integer values therebetween. In one embodiment, the invention includes a polynucleotide sequence encoding a functional luciferase (e.g., one that is capable of mediating the production of light in the presence of the appropriate substrate, for example, luciferin, under appropriate conditions), wherein the polynucleotide sequence comprises a fragment derived from SEQ ID NO: 3. Further, this aspect of the invention includes modifications of the polynucleotide sequence including, but not limited to, the following: codon optimization for expression in a selected cell type or organism (e.g., mice, Candida, or Cryptococcus); removal/modification of unwanted restriction sites; removal/modification of possible glycosylation sites; removal/modification of C-terminal peroxisome targeting sequences; removal/modification of transcription factor binding sites; removal/modification of palindromes; and/or removal/modification of RNA folding structures.

In another aspect, the invention includes expression cassettes comprising one or more transcriptional and/or translational control elements operably linked to any of the polynucleotides described herein.

In another aspect, the invention includes a host cell or transgenic animal comprising any of the polynucleotides described herein. In certain embodiments, the transgenic animal is a rodent (e.g., rat or mouse).

In yet another aspect, the invention includes a method for monitoring expression of a gene in a host cell, said method comprising monitoring the expression of luciferase in the host cell, said host cell comprising any expression cassette described herein.

In a still further aspect, a method for monitoring expression of a gene in a transgenic animal, said method comprising monitoring the expression of luciferase in the animal, said animal comprising any expression cassette described herein is provided.

In yet another aspect, the present invention comprises a polynucleotide, as described above, encoding a functional luciferase wherein the polynucleotide sequence is modified to optimize expression in a different, selected host system (e.g., plants, yeast, etc.). Further, the polynucleotide sequence may be modified to, for example, (i) disrupt transcriptional regulatory elements, and (ii) add or remove restriction sites.

These and other embodiments of the present invention will be apparent to those of skill in the art in view of the teachings herein.

### BRIEF DESCRIPTION OF THE DRAWINGS

FIG. 1 presents a modified nucleotide sequence (SEQ ID NO: 1) encoding a red railroad worm red luciferase according to the present invention. FIG. 1 also presents the corresponding amino acid coding sequence of the luciferase (SEQ ID NO: 2).

FIG. 2 is a comparison of the nucleotide sequence of the native railroad worm red luciferase-encoding sequence (labeled RRW red LUC native; SEQ ID NO: 3) and the modified sequence shown in FIG. 1 (labeled RRW red LUC optimized; SEQ ID NO: 1). Modified nucleotides are boxed and shaded. The parameters for the alignment were as follows: FAST algorithm, ktuple=2, gap penalty=5, window size=4, gap opening penalty=15, gap extension penalty=6.66.

FIG. 3 presents a native nucleotide sequence (SEQ ID NO: 3) encoding a red railroad worm red luciferase derived from Phrixothrix hirtus according to the present invention. FIG. 3 also presents the corresponding amino acid coding sequence of the luciferase (SEQ ID NO: 4).

### MODES FOR CARRYING OUT THE INVENTION

Throughout this application, various publications, patents, and published patent applications are referred to by an identifying citation. The disclosures of these publications, patents, and published patent specifications referenced in this application are hereby incorporated by reference into the present disclosure to more fully describe the state of the art to which this invention pertains.

The practice of the present invention will employ, unless otherwise indicated, conventional techniques of molecular biology, microbiology, cell biology and recombinant DNA, which are within the skill of the art. See, e.g., Sambrook, Fritsch, and Maniatis, MOLECULAR CLONING: A LABORATORY MANUAL, 2nd edition (1989); CURRENT PROTOCOLS IN MOLECULAR BIOLOGY, (F. M. Ausubel et al. eds., 1987); the series METHODS IN ENZYMOLOGY (Academic Press, Inc.); PCR 2: A PRACTICAL APPROACH (M. J. McPherson, B. D. Hames and G. R. Taylor eds., 1995); ANIMAL CELL CULTURE (R. I. Freshney. Ed., 1987); “Transgenic Animal Technology: A Laboratory Handbook,” by Carl A. Pinkert, (Editor) First Edition, Academic Press; ISBN: 0125571658; and “Manipulating the Mouse Embryo: A Laboratory Manual,” Brigid Hogan, et al., ISBN: 0879693843, Publisher: Cold Spring Harbor Laboratory Press, Pub. Date: September 1999, Second Edition.

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise. Thus, for example, reference to “a polypeptide” includes a mixture of two or more such agents.

Definitions

As used herein, certain terms will have specific meanings.

The terms “nucleic acid molecule” and “polynucleotide” are used interchangeably to and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term polynucleotide sequence is the alphabetical representation of a polynucleotide molecule. This alphabetical representation can be input into databases in a computer having a central processing unit and used for bioinformatics applications such as functional genomics and homology searching.

A “coding sequence” or a sequence which “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence. Thus, for example railroad worm luciferase can be codon optimized to represent preferred codon usage of mammalian gene sequences. “Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences which are immunologically identifiable with a polypeptide encoded by the sequence.

A “transcription factor” typically refers to a protein (or polypeptide) which affects the transcription, and accordingly the expression, of a specified gene. A transcription factor may refer to a single polypeptide transcription factor, one or more polypeptides acting sequentially or in concert, or a complex of polypeptides.

Typical “control elements” include, but are not limited to, transcription promoters, transcription enhancer elements, cis-acting transcription regulating elements (transcription regulators, e.g., a cis-acting element that affects the transcription of a gene, for example, a region of a promoter with which a transcription factor interacts to induce or repress expression of a gene), transcription initiation signals (e.g., TATA box), basal promoters, transcription termination signals, as well as polyadenylation sequences (located 3′ to the translation stop codon), sequences for optimization of initiation of translation (located 5′ to the coding sequence), translation enhancing sequences, and translation termination sequences. Transcription promoters can include, for example, inducible promoters (where expression of a polynucleotide sequence operably linked to the promoter is induced by an analyte, cofactor, regulatory protein, etc.), repressible promoters (where expression of a polynucleotide sequence operably linked to the promoter is induced by an analyte, cofactor, regulatory protein, etc.), and constitutive promoters.

“Expression enhancing sequences,” also referred to as “enhancer sequences” or “enhancers,” typically refer to control elements that improve transcription or translation of a polynucleotide relative to the expression level in the absence of such control elements (for example, promoters, promoter enhancers, enhancer elements, and translational enhancers (e.g., Shine and Delagarno sequences)).

The term “modulation” refers to both inhibition, including partial inhibition, as well as stimulation. Thus, for example, a compound that modulates expression of a reporter sequence may either inhibit that expression, either partially or completely, or stimulate expression of the sequence.

“Purified polynucleotide” refers to a polynucleotide of interest or fragment thereof which is essentially free, e.g., contains less than about 50%, preferably less than about 70%, and more preferably less than about 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

A “heterologous sequence” typically refers to either (i) a nucleic acid sequence that is not normally found in the cell or organism of interest, or (ii) a nucleic acid sequence introduced at a genomic site wherein the nucleic acid sequence does not normally occur in nature at that site. For example, a DNA sequence encoding a polypeptide can be obtained from yeast and introduced into a bacterial cell. In this case the yeast DNA sequence is “heterologous” to the native DNA of the bacterial cell. Alternatively, a promoter sequence, for example, from a Tie2 gene can be introduced into the genomic location of a fosB gene. In this case the Tie2 promoter sequence is “heterologous” to the native fosB genomic sequence.

A “polypeptide” is used in it broadest sense to refer to a compound of two or more subunit amino acids, amino acid analogs, or other peptidomimetics. The subunits may be linked by peptide bonds or by other bonds, for example ester, ether, etc. As used herein, the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is typically called a polypeptide or a protein. Amino acids are shown either by three letter or one letter abbreviations as follows:

| | Three Letter | One Letter |
|---|---|---|
| Amino Acid | Abbreviation | Abbreviation |
| Alanine | Ala | A |
| Cysteine | Cys | C |
| Aspartic Acid | Asp | D |
| Glutamic Acid | Glu | E |
| Phenylalanine | Phe | F |
| Glycine | Gly | G |
| Histidine | His | H |
| Isoleucine | Ile | I |
| Lysine | Lys | K |
| Leucine | Leu | L |
| Methionine | Met | M |
| Asparagine | Asn | N |
| Proline | Pro | P |
| Glutamine | Gln | Q |
| Arginine | Arg | R |
| Serine | Ser | S |
| Threonine | Thr | T |
| Valine | Val | V |
| Tryptophan | Trp | W |
| Tyrosine | Tyr | Y |

“Operably linked” refers to an arrangement of elements wherein the components so described are configured so as to perform their usual function. Thus, a given promoter that is operably linked to a coding sequence (e.g., a reporter expression cassette) is capable of effecting the expression of the coding sequence when the proper enzymes are present. The promoter or other control elements need not be contiguous with the coding sequence, so long as they function to direct the expression thereof. For example, intervening un-translated yet transcribed sequences can be present between the promoter sequence and the coding sequence and the promoter sequence can still be considered “operably linked” to the coding sequence.

“Recombinant” as used herein to describe a nucleic acid molecule means a polynucleotide of genomic, cDNA, semi-synthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used inter-changeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

An “isolated polynucleotide” molecule is a nucleic acid molecule separate and discrete from the whole organism with which the molecule is found in nature; or a nucleic acid molecule devoid, in whole or part, of sequences normally associated with it in nature; or a sequence, as it exists in nature, but having heterologous sequences (as defined below) in association therewith.

Techniques for determining nucleic acid and amino acid “sequence identity” also are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6): 6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

One of skill in the art can readily determine the proper search parameters to use for a given sequence in the above programs. For example, the search parameters may vary based on the size of the sequence in question. Thus, for example, a representative embodiment of the present invention would include a polynucleotide comprising X contiguous nucleotides wherein (i) the X contiguous nucleotides have at least about a selected level of percent identity relative to Y contiguous nucleotides of one or more of the sequences described herein or fragment thereof, and (ii) for search purposes X equals Y, wherein Y is a selected reference polynucleotide of defined length (for example, a length of from 15 nucleotides up to the number of nucleotides present in a selected full-length sequence, e.g., SEQ ID NO: 1, 1641 nucleotides, including all integer values falling within the above-described ranges. A “fragment” of a polynucleotide refers to any length polynucleotide molecule derived from a larger polynucleotide described herein (i.e., Y contiguous nucleotides, where X=Y as just described). Exemplary fragment lengths include, but are not limited to, at least about 6 contiguous nucleotides, at least about 50 contiguous nucleotides, about 100 contiguous nucleotides, about 250 contiguous nucleotides, about 500 contiguous nucleotides, or at least about 1000 contiguous nucleotides or more, wherein such contiguous nucleotides are derived from a larger sequence of contiguous nucleotides.

The purified polynucleotides and polynucleotides used in construction of expression cassettes of the present invention include the sequences disclosed herein as well as related polynucleotide sequences having sequence identity of approximately 80% to 100% and integer values therebetween. Typically the percent identities between the sequences disclosed herein and the claimed sequences are at least about 85-90%, preferably at least about 90-95%, more preferably at least about 95-98%, and most preferably at least about 98-100% sequence identity (including all integer values falling within these described ranges). These percent identities are, for example, relative to the claimed sequences, or other sequences of the present invention, when the sequences of the present invention are used as the query sequence.

Alternatively, the degree of sequence similarity between polynucleotides can be determined by hybridization of polynucleotides under conditions that form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80%-100% or any integer value therebetween, preferably at least about 85%-90%, more preferably at least about 90%-95%, more preferably at least about 95%-98%, and even more preferably 98%-100% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The degree of sequence identity between two nucleic acid molecules affects the efficiency and strength of hybridization events between such molecules. A partially identical nucleic acid sequence will at least partially inhibit a completely identical sequence from hybridizing to a target molecule. Inhibition of hybridization of the completely identical sequence can be assessed using hybridization assays that are well known in the art (e.g., Southern blot, Northern blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. A nucleic acid molecule that is capable of hybridizing selectively to a target sequence under “moderately stringent” typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.).

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

“Nucleic acid expression vector” or “expression cassette” refers to an assembly that is capable of directing the expression of a sequence or gene of interest. The nucleic acid expression vector includes a promoter that is operably linked to the sequences or gene(s) of interest. Other control elements may be present as well. Expression cassettes described herein may be contained within a plasmid construct. In addition to the components of the expression cassette, the plasmid construct may also include a bacterial origin of replication, one or more selectable markers, a signal which allows the plasmid construct to exist as single-stranded DNA (e.g., a M13 origin of replication), a multiple cloning site, and a “mammalian” origin of replication (e.g., a SV40 or adenovirus origin of replication).

An “expression cassette” comprises any nucleic acid construct capable of directing the expression of a gene/coding sequence of interest. Such cassettes can be constructed into a “vector,” “vector construct,” “expression vector,” or “gene transfer vector,” in order to transfer the expression cassette into target cells. Thus, the term includes cloning and expression vehicles, as well as viral vectors.

A “light generating protein” or “light-emitting protein” is a bioluminescent or fluorescent protein capable of producing light typically in the range of 200 nm to 1100 nm, preferably in the visible spectrum (i.e., between approximately 350 nm and 800 nm). Bioluminescent proteins produce light through a chemical reaction (typically requiring a substrate, energy source, and oxygen). Fluorescent proteins produce light through the absorption and re-emission of radiation (such as with green fluorescent protein). Examples of bioluminescent proteins include, but are not limited to, the following: “luciferase,” unless stated otherwise, includes procaryotic (e.g., bacterial lux-encoded) and eucaryotic (e.g., firefly luc-encoded) luciferases, as well as variants possessing varied or altered optical properties, such as luciferases that produce different colors of light (e.g., Kajiyama, N., and Nakano, E., Protein Engineering 4(6): 691-693 (1991)); and “photoproteins,” for example, calcium activated photoproteins (e.g., Lewis, J. C., et al., Fresenius J. Anal. Chem. 366(6-7): 760-768 (2000)). Examples of fluorescent proteins include, but are not limited to, green, yellow, cyan, blue, and red fluorescent proteins (e.g., Hadjantonakis, A. K., et al., Histochem. Cell Biol. 115(1): 49-58 (2001)).

“Bioluminescent protein substrate” describes a substrate of a light-generating protein, e.g., luciferase enzyme, that generates an energetically decayed substrate (e.g., luciferin) and a photon of light typically with the addition of an energy source, such as ATP or FMNH2, and oxygen. Examples of such substrates include, but are not limited to, decanal in the bacterial lux system, 4,5-dihydro-2-(6-hydroxy-2-benzothiazolyl)-4-thiazolecarboxylic acid (or simply called luciferin) in the Firefly luciferase (luc) system, “panal” in the bioluminescent fungus Panellus stipticus system (Tetrahedron 44:1597-1602, 1988) and N-iso-valeryl-3-aminopropanol in the earth worm Diplocardia longa system (Biochem. 15:1001-1004, 1976). In some systems, aldehyde can be used as a substrate for the light-generating protein.

“Light” is defined herein, unless stated otherwise, as electromagnetic radiation having a wavelength of between about 200 nm (e.g., for UV-C) and about 1100 nm (e.g., infrared). The wavelength of visible light ranges between approximately 350 nm to approximately 800 nm (i.e., between about 3,500 angstroms and about 8,000 angstroms).

“Animal” as used herein typically refers to a non-human mammal, including, without limitation, farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

A “transgenic animal” refers to a genetically engineered animal or offspring of genetically engineered animals. A transgenic animal usually contains material from at least one unrelated organism, such as from a virus, plant, or other animal. The “non-human animals” of the invention include vertebrates such as rodents, non-human primates, sheep, dogs, cows, amphibians, birds, fish, insects, reptiles, etc. The term “chimeric animal” is used to refer to animals in which the heterologous gene is found, or in which the heterologous gene is expressed in some but not all cells of the animal.

A “gene” as used in the context of the present invention is a sequence of nucleotides in a genetic nucleic acid (chromosome, plasmid, etc.) with which a genetic function is associated. A gene is a hereditary unit, for example of an organism, comprising a polynucleotide sequence (e.g., a DNA sequence for mammals) that occupies a specific physical location (a “gene locus” or “genetic locus”) within the genome of an organism. A gene can encode an expressed product, such as a polypeptide or a polynucleotide (e.g., tRNA). Alternatively, a gene may define a genomic location for a particular event/function, such as the binding of proteins and/or nucleic acids (e.g., phage attachment sites), wherein the gene does not encode an expressed product. Typically, a gene includes coding sequences, such as, polypeptide encoding sequences, and non-coding sequences, such as, promoter sequences, poly-adenlyation sequences, transcriptional regulatory sequences (e.g., enhancer sequences). Many eucaryotic genes have “exons” (coding sequences) interrupted by “introns” (non-coding sequences). In certain cases, a gene may share sequences with another gene(s) (e.g., overlapping genes). It is noted that in the general population, wild-type genes may include multiple prevalent versions that contain alterations in sequence relative to each other and yet do not cause a discernible pathological effect. These variations are designated “polymorphisms” or “allelic variations.”

Before describing the present invention in detail, it is to be understood that this invention is not limited to particular formulations or method parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

General Overview

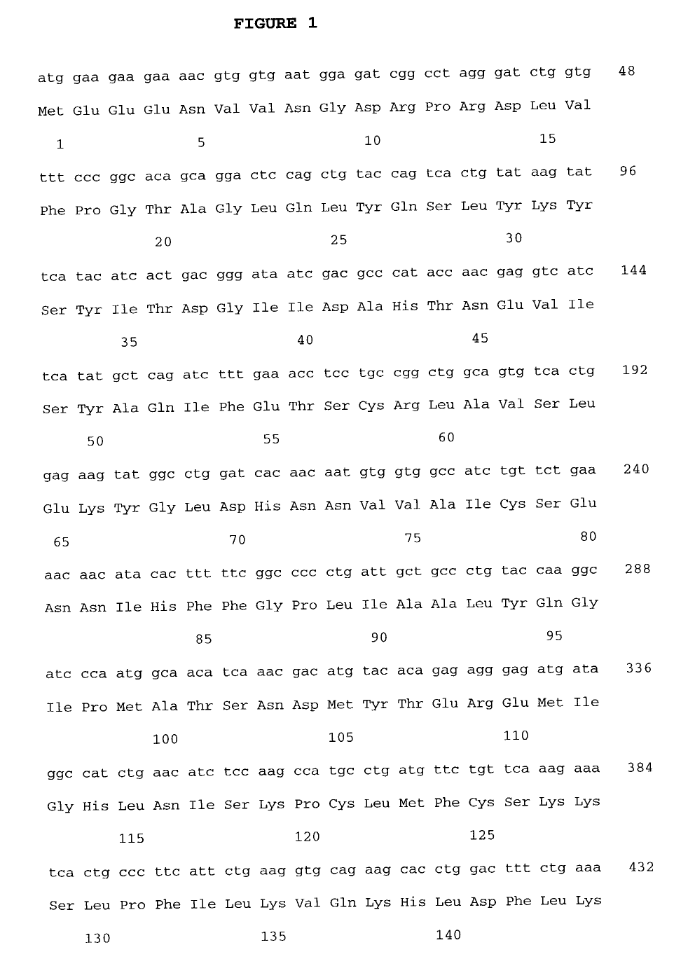

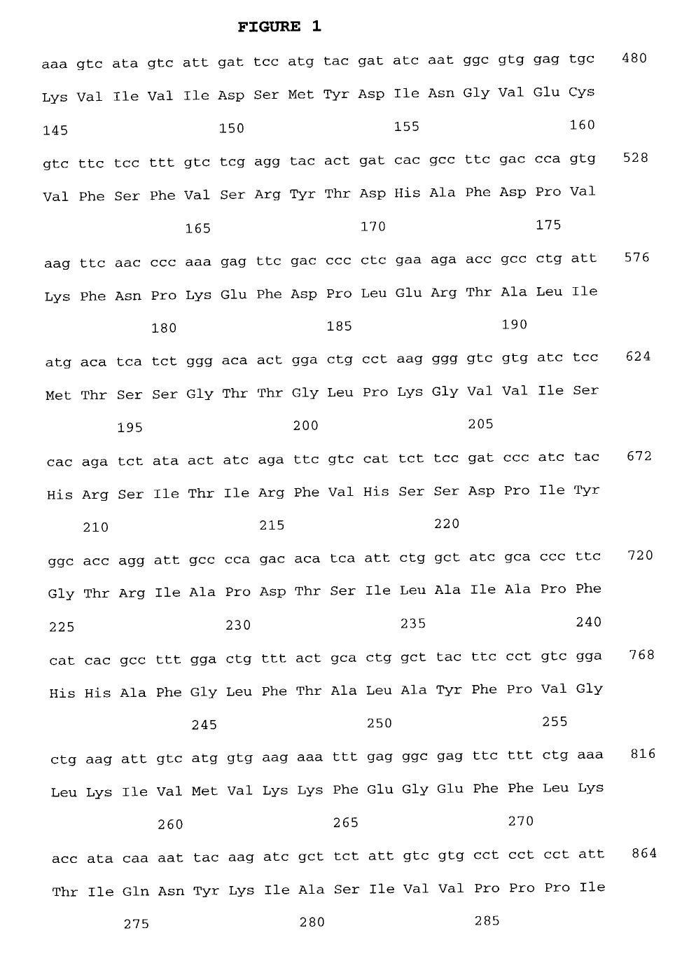

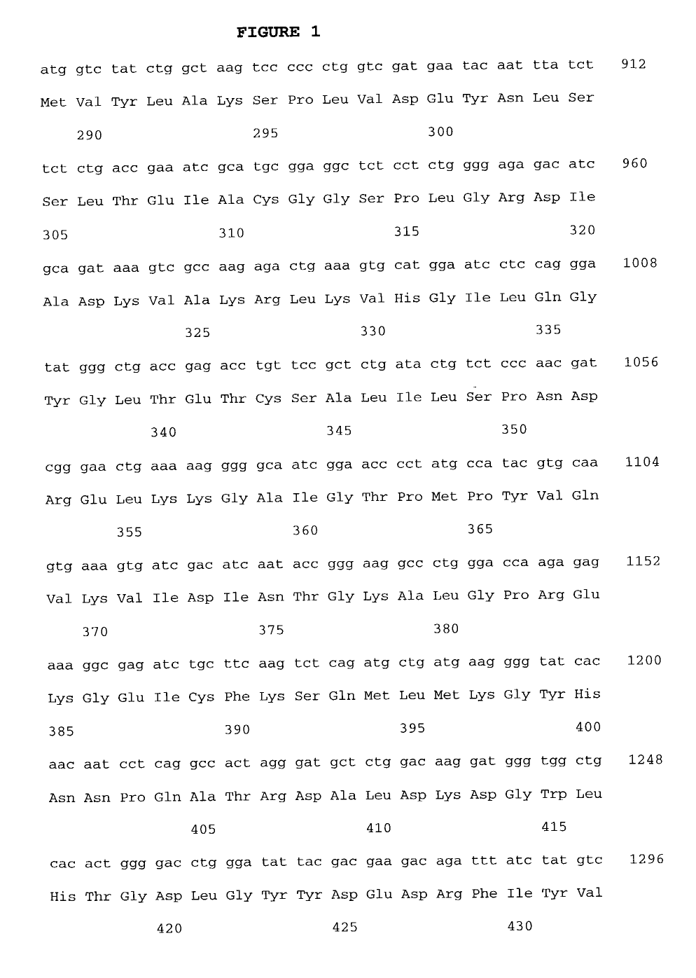

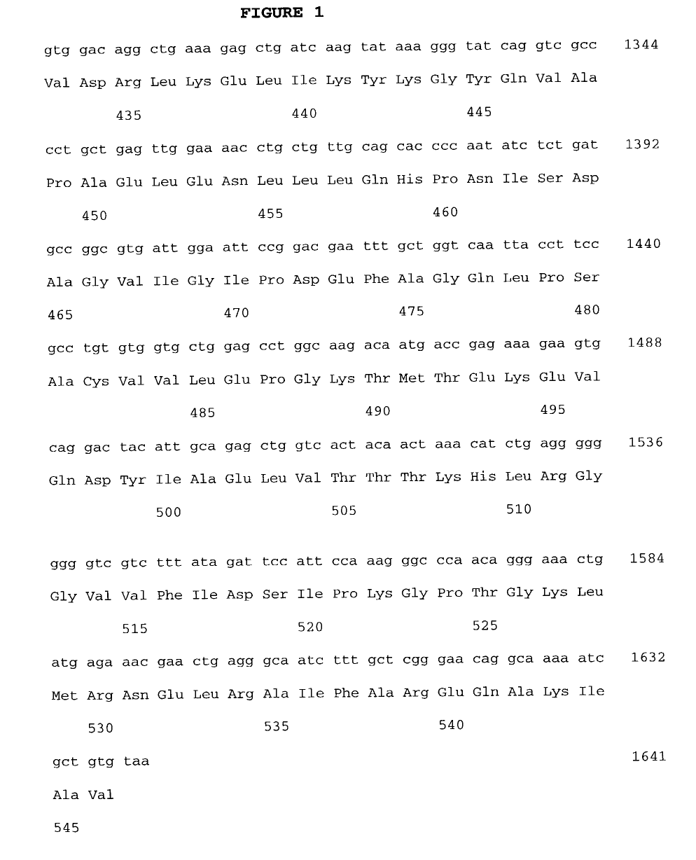

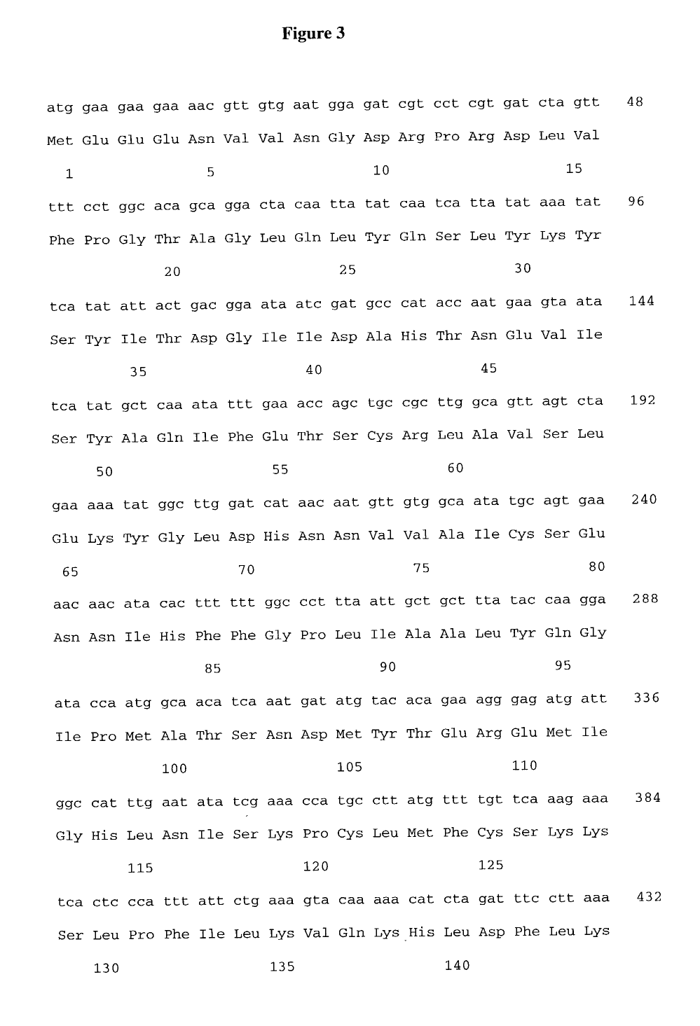

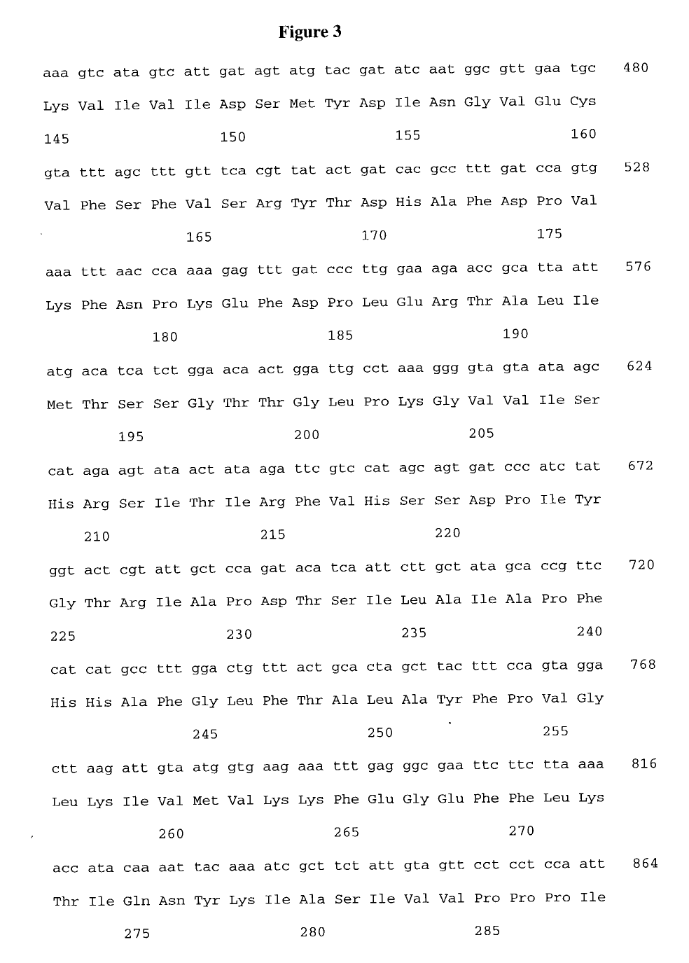

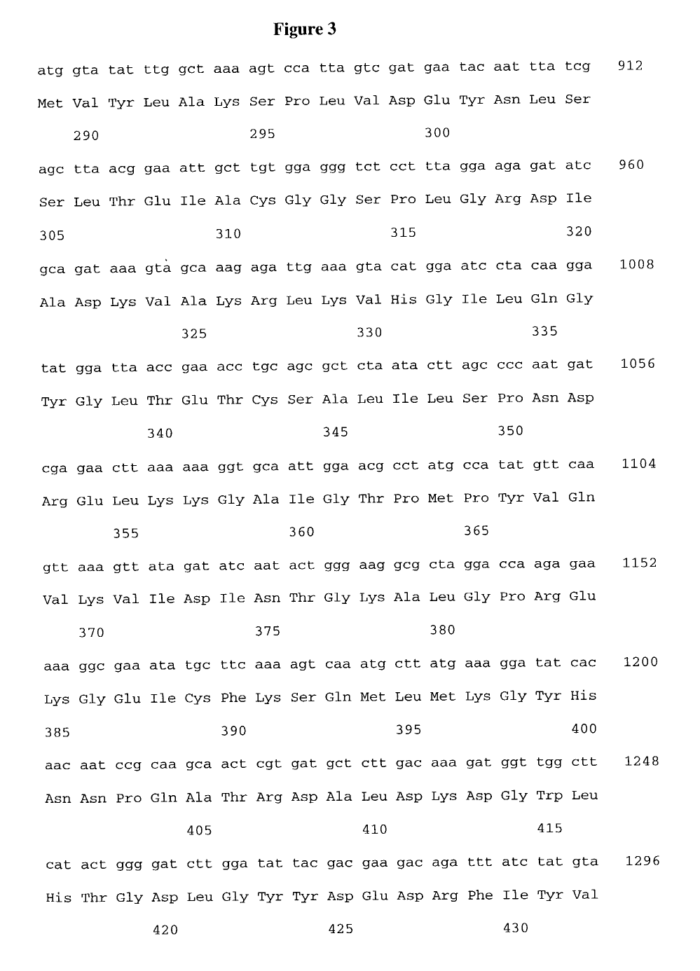

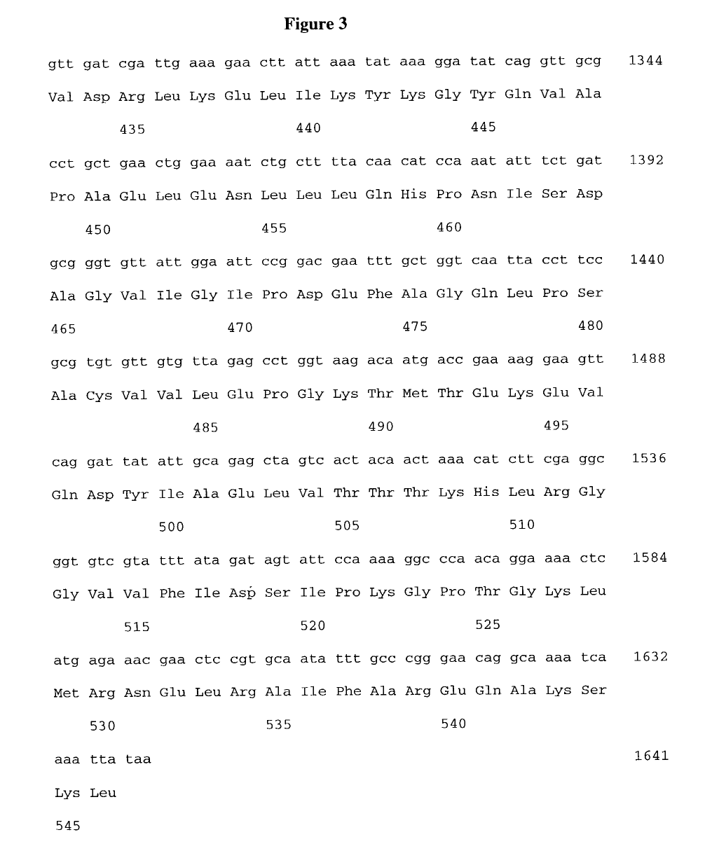

Described herein are native and modified forms of railroard worm red luciferase. The native coding sequence was derived from Phrixothrix hirtus. The present invention is directed to sequences encoding functional (e.g., able to mediate the production of light under appropriate conditions) red luciferase of Phrixothrix hirtus. Native polynucleotide and polypeptide red luciferase sequences (SEQ ID NO: 3 and SEQ ID NO: 4, respectively), as well as modified, optimized polynucleotide and polypeptide sequences (SEQ ID NO: 1 and SEQ ID NO: 2, respectively) are taught herein. In one aspect, the invention comprises an isolated polynucleotide or polypeptide having at least about 85% sequence identity to the sequences shown in FIG. 1 (SEQ ID NO: 1 and SEQ ID NO: 2) or fragments thereof. In another aspect, the invention comprises an isolated polynucleotide or polypeptide having at least about 85% sequence identity to the sequences shown in FIG. 3 (SEQ ID NO: 3 and SEQ ID NO: 4) or fragments thereof. Preferably, the sequences exhibit at least about 90% sequence identity, more preferably 95% sequence identity, and most preferably 98% sequence identity to the sequences described herein. In certain embodiments, the isolated polynucleotide sequence comprises a polynucleotide consisting of full-length SEQ ID NO: 1 and/or SEQ ID NO: 3. In certain embodiments, the isolated polypeptide sequence comprises a polypeptide consisting of full-length SEQ ID NO: 2 and/or SEQ ID NO: 4. In other embodiments, the sequences of the present invention can include fragments of the polynucleotides described herein, for example, from about 15 nucleotides up to the number of nucleotides present in the full-length sequences described herein (e.g., see the Sequence Listing and Figures), including all integer values falling within the above-described range. For example, fragments of the polynucleotide sequences of the present invention may be 30-60 nucleotides, 60-120 nucleotides, 120-240 nucleotides, 240-480 nucleotides, 480-1000 nucleotides, 1000 to 1641 nucleotides, and all integer values therebetween. In one embodiment, the invention includes a polynucleotide sequence encoding a functional luciferase (i.e., one that is capable of mediating the production of light, for example, in the presence of the appropriate substrate under appropriate conditions), wherein the polynucleotide sequence comprises a fragment. Further, this aspect of the invention includes modifications of the polynucleotide sequences encoding polypeptide sequences including, but not limited to, the following: codon optimization for expression in a selected cell type or organism (for example, human, rodent (e.g., mouse), Candida, or Cryptococcus); removal/modification of unwanted restriction sites; removal/modification of possible glycosylation sites; removal/modification of C-terminal peroxisome targeting sequences; removal/modification of transcription factor binding sites; removal/modification of palindromes; and/or removal/modification of RNA folding structures. The invention also includes polypeptides encoded by the above-described polynucleotides or fragments thereof.

Unlike the most widely studied and modified luciferase gene, which is derived from the firefly Photinus pyralis, modifications of RR red luciferase have not heretofore been described. These novel sequences are useful in a wide variety of applications, including all applications where luciferase is used as a reporter gene. Advantages of the present invention include, but are not limited, to (1) increasing expression of RR red luciferase in host cells (in vivo and in vitro), for instance by optimizing codon usage to reflect that of the host cell; (2) obtaining expression of RR red luciferase that is unbiased by peroxisomal physiology; (3) obtaining a reporter gene that is genetically neutral in that it contains no major genetic regulatory elements, palindromic sequences and/or RNA structures (e.g., hairpins) that interfere with expression; and (4) obtaining a luciferase that provides reliability and convenience in diverse applications.

Isolation and Sequencing of the Native Railroad Worm Red Luciferase

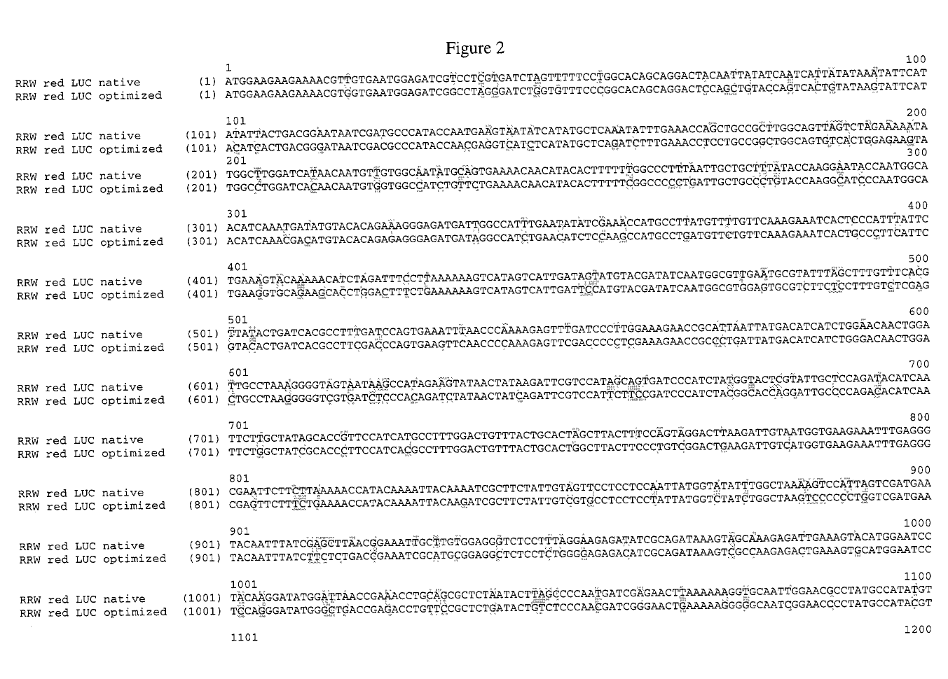

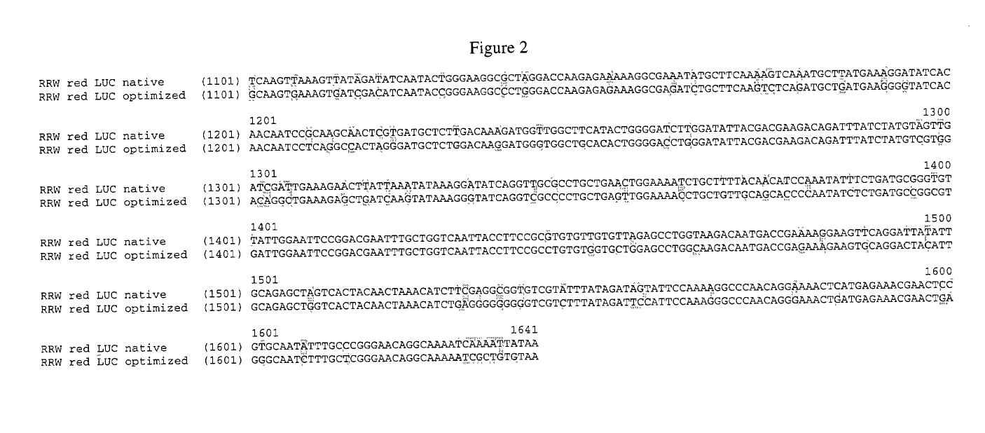

Originally the starting sequence for optimization was the sequence presented as GENBANK Accession No. AF139645, which was based on the sequence of a cloned cDNA molecule (Ph_{RE}, described in Viviani, V. R., et al., Biochemistry 38:8271-8279, 1999). The originally optimized sequence was designated RRLUCX. However, the RRLUCX sequence did not encode a polypeptide that produced light. The original clone (Ph_{RE}) was independently sequenced and several sequence errors were discovered relative to the AF139645 sequence. The correct sequence of the original clone is presented in the top line of FIG. 2 (SEQ ID NO: 3) and in FIG. 3.

Modifications to Railroad Worm Red Luciferase

To improve the general suitability of luciferase in molecular biological applications, a modified form of the luciferase gene from the Phrixothrix hirtus (railroad worm or RR) has been developed. The Phrixothrix hirtus larva produces both a green and red luciferase (see, Viviani et al. (1999) Biochemistry 38(26): 8271-8279, herein incorporated by reference).

A railroad worm red luciferase was modified to optimize expression in mammalian cells. An exemplary modified luciferase-encoding sequence is shown in FIG. 1 (SEQ ID NO: 1) and FIG. 2 (RRW red LUC optimized). An polypeptide translation of SEQ ID NO: 1 is also presented in FIG. 1. This modified luciferase was obtained using one or more of the following procedures: (a) codon optimization to match usage in mammalian genes, preferably without changing the amino acid sequence of the protein; (b) removal of unwanted restriction enzyme sites, preferably without changing the amino acid sequence; (c) removal of peroxisome targeting sequence (SKL) at the end of the protein; (d) removal of as many as possible putative transcription factor binding sites; (e) removal of palindromes and repeats in the DNA sequence; and (f) checking the mRNA for secondary structure problems (e.g., large hairpins, etc.). In addition, the sequence can be modified to remove possible glycosylation sites (e.g., Asn-X-Ser/Thr).

The sequence to be modified can be any railroad worm luciferase-encoding sequence, for example the sequence shown in FIG. 2, labeled RRW red LUC native. A preferred method of site-specifically mutating the starting sequence (e.g., any railroad worm red luciferase-encoding sequence) is by using PCR. General procedures for PCR as taught in MacPherson et al., PCR: A PRACTICAL APPROACH, (IRL Press at Oxford University Press, (1991)). PCR conditions for each application reaction may be empirically determined. A number of parameters influence the success of a reaction. Among these parameters are annealing temperature and time, extension time, Mg2+ and ATP concentration, pH, and the relative concentration of primers, templates and deoxyribonucleotides. After amplification, the resulting fragments can be detected by agarose gel electrophoresis followed by visualization with ethidium bromide staining and ultraviolet illumination.

Site-specific mutagenesis can also be performed using techniques known in the art, for example using the QuikChange® kit (Stratagene, La Jolla, Calif.) and following the manufacturer's directions. Site-directed mutagenesis against single-stranded plasmid templates is described for example in Lewis et al. (1990) Nuc. Acids Res. 18:3439-3443. According to this method, a mutagenic primer designed to correct a defective ampicillin resistance gene is used in combination with one or more primers designed to mutate discreet regions within the target gene. Rescued antibiotic resistance coupled with distant non-selectable mutations in the target gene results in high frequency capture of the desired mutations.

Another method for obtaining optimized railroad worm red luciferase is random mutagenesis to randomly alter the amino acids, followed by screening for clones exhibiting efficient luminescence. Random mutagenesis can be performed, for example, by generating oligonucleotide(s) to randomly alter the target DNA sequence, for example the peroxisome targeting sequence (SKL) at the C-terminus of luciferase. DNA containing a population of random C-terminal mutations is used to transform E. coli cells and ampicillin resistant colonies can be screen for bioluminescence by any method known in the art. Those clones selected for high luciferase expression can then be sequenced and otherwise analyzed for amino acid sequence deviation from the natural peroxisome targeting sequence.

1. Codon Optimization

Codon optimization can be achieved, for example, by utilizing the Codon Usage Database, available on the World Wide Web at http://www.kazusa.or.jp/codon/. Codon usage tables were generated from human, mouse, Candida and Cryptococcus coding sequences. This database was generated using the coding sequences located in Genbank. Comparing mouse and human codon usage, they are almost identical, varying by <5% for each codon. Therefore, the construct made should work in both organisms. The Cryptococcus codon use is similar (≦10%) to that of mammalian cells for about three quarters (75%) of the amino acids. In Candida, the codon usage is generally the opposite of that the other organisms and, therefore, the construct would have to be made for optimal codon usage.

Using a codon usage chart for human genes, the RR red luciferase was modified so as to bring the codons close to the percentages used in mammals. Table 1 shows the original number of amino acid residues (column: Amino Acid) and codons used (column: Codon) present in the native protein (column: orig #), and in the modified, optimized sequence (column: new#). Also, the percent of each different codon used for each given amino acid is presented for the native sequence (column: orig %), and the modified, optimized sequence (column: new %). Further, the percent of each different codon used for each given amino acid is presented for typical coding sequences in human genes (column: % in human genes), mouse genes (column: % in mice), Candida genes (column: % in Candida), and Cryptococcus genes (column: % in Crypto).

TABLE 1

| Amino | | orig | orig | new | new | % in human | % in | % in | % in |
|---|---|---|---|---|---|---|---|---|---|
| Acid | Codon | # | % | # | % | genes | mice | Candida | Crypto |
| Met | ATG | 14 | 100 | 14 | 100 | 100 | 100 | 100 | 100 |
| Trp | TGG | 1 | 100 | 1 | 100 | 100 | 100 | 100 | 100 |
| Glu | GAA | 26 | 84 | 15 | 48 | 41 | 40 | 81 | 46 |
| | GAG | 5 | 16 | 16 | 52 | 59 | 60 | 19 | 54 |
| Phe | TTT | 19 | 76 | 13 | 52 | 44 | 43 | 64 | 34 |
| | TTC | 6 | 24 | 12 | 48 | 56 | 57 | 32 | 66 |
| Asp | GAT | 25 | 83 | 14 | 47 | 46 | 44 | 75 | 47 |
| | GAC | 5 | 17 | 16 | 53 | 54 | 56 | 25 | 53 |
| Cys | TGT | 3 | 33 | 4 | 44 | 45 | 46 | 84 | 49 |
| | TGC | 6 | 67 | 5 | 56 | 55 | 54 | 16 | 51 |
| His | CAT | 12 | 80 | 6 | 40 | 41 | 39 | 71 | 40 |
| | CAC | 3 | 20 | 9 | 60 | 59 | 61 | 29 | 60 |
| Gln | CAA | 12 | 80 | 4 | 27 | 26 | 25 | 84 | 45 |
| | CAG | 3 | 20 | 11 | 73 | 74 | 75 | 16 | 55 |
| Asn | AAT | 13 | 65 | 8 | 40 | 46 | 42 | 67 | 37 |
| | AAC | 7 | 35 | 12 | 60 | 54 | 58 | 33 | 63 |
| Tyr | TAT | 17 | 71 | 11 | 46 | 43 | 41 | 67 | 32 |
| | TAC | 7 | 29 | 13 | 54 | 57 | 59 | 33 | 68 |
| Lys | AAA | 32 | 82 | 17 | 45 | 42 | 38 | 72 | 28 |
| | AAG | 7 | 18 | 21 | 55 | 58 | 62 | 28 | 72 |
| Ile | ATT | 19 | 42 | 13 | 28 | 35 | 33 | 60 | 38 |
| | ATC | 8 | 18 | 25 | 54 | 49 | 52 | 21 | 55 |
| | ATA | 18 | 40 | 8 | 17 | 16 | 15 | 19 | 7 |
| Pro | CCT | 11 | 35 | 12 | 39 | 28 | 30 | 27 | 42 |
| | CCC | 3 | 10 | 10 | 32 | 33 | 31 | 8 | 31 |
| | CCA | 14 | 45 | 8 | 26 | 27 | 28 | 58 | 15 |
| | CCG | 3 | 10 | 1 | 3 | 12 | 11 | 5 | 12 |
| Thr | ACT | 11 | 38 | 9 | 31 | 24 | 24 | 47 | 32 |
| | ACC | 7 | 24 | 11 | 38 | 36 | 36 | 21 | 40 |
| | ACA | 9 | 31 | 9 | 31 | 28 | 29 | 27 | 15 |
| | ACG | 2 | 7 | 0 | 0 | 12 | 11 | 5 | 13 |
| Ala | GCT | 13 | 37 | 12 | 33 | 26 | 29 | 50 | 32 |
| | GCC | 4 | 11 | 13 | 36 | 40 | 39 | 21 | 38 |
| | GCA | 14 | 40 | 11 | 31 | 23 | 22 | 25 | 13 |
| | GCG | 4 | 11 | 0 | 0 | 11 | 10 | 4 | 17 |
| Gly | GGT | 7 | 18 | 1 | 3 | 16 | 17 | 56 | 46 |
| | GGC | 9 | 23 | 13 | 33 | 34 | 34 | 8 | 31 |
| | GGA | 20 | 50 | 12 | 24 | 25 | 26 | 23 | 16 |
| | GGG | 4 | 10 | 14 | 35 | 25 | 23 | 13 | 11 |
| Val | GTT | 15 | 38 | 0 | 0 | 18 | 17 | 54 | 28 |
| | GTC | 6 | 15 | 18 | 45 | 24 | 25 | 17 | 42 |
| | GTA | 13 | 33 | 0 | 0 | 11 | 11 | 13 | 11 |
| | GTG | 5 | 13 | 22 | 55 | 47 | 47 | 16 | 18 |
| Arg | CGT | 6 | 30 | 0 | 0 | 8 | 9 | 16 | 17 |
| | CGC | 1 | 5 | 0 | 0 | 19 | 18 | 2 | 11 |
| | CGA | 3 | 15 | 0 | 0 | 11 | 12 | 10 | 25 |
| | CGG | 1 | 5 | 4 | 20 | 22 | 19 | 2 | 8 |
| | AGA | 8 | 40 | 8 | 40 | 20 | 21 | 65 | 16 |
| | AGG | 1 | 5 | 8 | 40 | 20 | 21 | 7 | 22 |
| Ser | TCT | 4 | 13 | 11 | 35 | 18 | 19 | 27 | 29 |
| | TCC | 1 | 3 | 10 | 32 | 22 | 22 | 12 | 25 |
| | TCA | 10 | 31 | 9 | 29 | 15 | 14 | 29 | 12 |
| | TCG | 2 | 6 | 1 | 3 | 6 | 6 | 8 | 10 |
| | AGT | 8 | 25 | 0 | 0 | 15 | 15 | 19 | 10 |
| | AGC | 7 | 22 | 0 | 0 | 25 | 25 | 5 | 15 |
| Leu | CTT | 13 | 25 | 0 | 0 | 13 | 13 | 11 | 28 |
| | CTC | 3 | 6 | 3 | 6 | 20 | 20 | 3 | 29 |
| | CTA | 9 | 17 | 0 | 0 | 7 | 8 | 4 | 3 |
| | CTG | 4 | 8 | 44 | 86 | 40 | 40 | 3 | 10 |
| | TTA | 15 | 29 | 2 | 4 | 7 | 6 | 38 | 6 |
| | TTG | 8 | 15 | 2 | 4 | 13 | 13 | 40 | 23 |

In preparing modified railroad worm luciferase, it is preferable to change all of the leucine codons to CTG, as CTG is the most used leucine codon in mammalian cells. However, less than all of these codons can also be changed. Furthermore, leucine (or other codons) can also be changed to other codons to remove restriction sites and transcription factor binding sites.

2. Removal of Unwanted Restriction Enzyme Sites

The restriction enzyme sites in the RR red gene can be mapped to identify and/or remove unwanted restriction enzyme sites. Such modifications can be done prior to, after or independent of the other modifications described herein (codon optimization, etc.). In one embodiment described herein, a single Sma I, and two Pst I sites were located in the gene following codon optimization. One of the PstI sites was introduced during codon optimization. Accordingly, nucleotides 69 and 1002 of SEQ ID NO: 1 were modified to disrupt the two PstI sites, and nucleotide 1614 of SEQ ID NO: 1 was modified to disrupt the Sma I site, each without changing the amino acid sequence.

For ease in cloning, restriction sites are preferably added to the 5′ and 3′ end of the luciferase-encoding sequence. Preferably, these restriction sites are unique. If, however, the added restriction sites are also found internally, the internal site can be modified without affecting the amino acid sequence. For example, if the nucleotides CC are added immediately before the start codon (at the 5′ end), a NcoI site is created (CCATGG). Such internal sites may be undesirable and can be readily modified following the teachings described herein (e.g., nucleotide 990 of SEQ ID NO: 1 was modified to removal an internal NcoI site).

3. Removal of Possible Glycosylation Sites

Native luciferase expressed in the peroxisomes or the cytosol is not typically post-translationally modified. However, in certain applications, for example applications in which the modified luciferase is used as part of a fusion protein and is excreted, the resulting polypeptide may be directed into the endoplasmic reticulum or Golgi apparatus where post-translational modification such as N-linked glycosylation are known to occur. Because such post-translational modifications may affect luciferase expression, it may be desirable in these instances to remove possible glycosylation sites.

There are two possible glycosylation sites in RR red (Asn-X-Ser/Thr). They are both N—I—S sites and are located at amino acids 116-118 (nucleotides 347-355) and 461-463 (nucleotides 1381-1389). None, one or both of these sites may be altered, for example, by modifying the asparagine (aa 461) to aspartic acid.

4. Removal of C-terminal Peroxisome Targeting Sequence

A major concern in the use of the native luciferases as genetic reporters is potential intracellular partitioning into peroxisomes. The presence of this foreign protein in peroxisomes, and moreover, the resulting competition with native host proteins for peroxisomal transport has undefined affects on the normal cellular physiology. Variable subcellular localization of luciferase also compromises its value as a quantitative marker of gene activity. These potential problems reduce the general reliability of luciferase in reporter applications. Thus, it may be desirable to remove or render non-functional the peroxisome targeting sequence.

In RR red luciferase, a peroxisome targeting sequence (Ser-Lys-Leu) is located at the end of the gene. In certain aspects, this sequence is changed to encode Ile-Ala-Val by modifying native nucleotides 1630 through 1637 of SEQ ID NO: 3 from TCAAAAT to ATCGCTG.

5. Removal of Transcription Factor Binding Sites

Any gene may contain regulatory sequences within its coding region which could mediate genetic activity through native regulatory function or via recognition by transcription factors in a foreign host. These sequences may alter expression of luciferase and were, therefore, altered while keeping the codon usage optimal and without affecting the amino acid sequence.

A table of 312 transcription factor binding sites is available in the program MacDNASIS. The RR luc sequence was analyzed for these sites and as many as possible were removed.

6. Removal of Palindromes

Palindromic sequences can affect expression. Using web-based programs, the gene sequence was searched for inverted repeats, tandem repeats, and palindromes. No inverted or tandem repeats of significant size were found. No perfect palindromes of over 9 bp were found and only one palindrome of 10 bp and one of 9 bp were found when one mismatch was allowed. These sequences were not altered.

Subsequently, using a web based program, the sequence was searched for DNA sequences repeated in the genome of primates (e.g., Alu sequences), rodents or other mammals. None were found.

7. RNA Folding Structures

Using the mfold3.0 program located at the Macfarlane Burnet Center in Australia (http://mfold.burnet.edu.au), several RNA folding structures were plotted. Upon inspection of the hairpins or base paired regions plotted, there were no large regions (>6 bases) of Gs and Cs in the base paired regions. They were either evenly divided between G-C and A-U pairs or mostly A-U pairs.

8. Summary of Modifications to the RRLUCX Sequence

As discussed above, the original starting sequence for optimization was the sequence presented as GENBANK Accession No. AF139645, which was based on the sequence of a cloned cDNA molecule (Ph_{RE}, described in Viviani, V. R., et al., Biochemistry 38:8271-8279, 1999). The originally optimized sequence was designated RRLUCX. However, the RRLUCX sequence did not produce light.

Table 2 is a summary of the nucleic acid modifications made to the RRLUCX sequence in order to obtain the optimized, modified Red Railroad Worm luciferase sequence (labeled “RRLUCXC” in Table 2, and “RRW red LUC optimized” in FIG. 2). The nucleotide (SEQ ID NO: 1) and protein (SEQ ID NO: 2) sequences of the RRW red LUC modified, optimized sequence are presented in FIG. 1. FIG. 2 presents a nucleotide sequence comparison between the native Red Railroad Worm luciferase (SEQ ID NO: 3) and the RRW red LUC optimized sequence (SEQ ID NO: 1).

The RRW red LUC optimized (RRLUCXC) sequence was completely functional when expressed in host cells and produced a light of λₘₐₓ approximately 622 nm.

TABLE 2

| Purpose | Construct | Position | Modification |
|---|---|---|---|
| Arg145-Lys | RRLUCX | 418 | CTGGACTTTCTGAAAAGAGTCATAGTC |
| | RRLUCXC | | CTGGACTTTCTGAAAAAAGTCATAGTC |
| Asp165-Val | RRLUCX | 474 | GGAGTGCGTCTTCTCCTTTGATTCGAGGAACACTGATCACGCCTTCG |
| & Arg168-Tyr | RRLUCXC | | GGAGTGCGTCTTCTCCTTTGTCTCGAGGTACACTGATCACGCCTTCG |
| & XhoI site | | | |
| introduction | | | |
| Cys303-Leu | RRLUCX | 891 | GGTCGATGAATACAATTGCT*GCTTCCGGAGGCTCTCCTCTGG |
| & Ser311-Cys | RRLUCXC | | GGTCGATGAATACAATTTAT*GCATGCGGAGGCTCTCCTCTGG |
| & SphI site | | | |
| introduction | | | where * is CTTCTCTGACCGAAATC |
| Frameshift | RRLUCX | 1390 | GAT*T-GAGTTCCGGACAAACCTGCTGGTCAATTACCTGTCCGCCTGTGTGGTG |
| aa 496-480 | RRLUCXC | | |
| | | | GAT*TGGAATTCCGGACGAATTTGCTGGTCAATTACCT-TCCGCCTGTGTGGTG |
| | | | where * is GCCGGCGTGAT |

Applications

The railroad worm red luciferase sequences described herein find use in a wide variety of procedures and applications. The native, native-modified, optimized, and/or modified-optimized red luciferases can, for example, be employed as described herein below.

The isolated polynucleotides of the present invention may be incorporated into expression cassettes. The expression cassettes described herein may typically include the following components: (1) a polynucleotide comprising a first polynucleotide, for example, having at least about 85-100% sequence identity to SEQ ID NO: 1 or SEQ ID NO: 3, wherein said first polynucleotide encodes a polypeptide capable of mediating light-production in the presence of an appropriate substrate, e.g., luciferin, under appropriate conditions, (2) a transcription control element operably linked to the polynucleotide, wherein the control element is heterologous to the coding sequences of the light generating protein. Transcription control elements may be associated with, for example, a basal transcription promoter to confer regulation provided by such control elements on such a basal transcription promoter.

The present invention also includes providing such expression cassettes in vectors, comprising, for example, a suitable vector backbone and optionally a sequence encoding a selection marker e.g., a positive or negative selection marker. Vectors carrying sequences encoding a red luciferase of the present invention, encoding fusions of a red luciferase and one or more additional polypeptides, or comprising further coding sequences can be constructed. The vectors carrying a red luciferase can be constructed utilizing methodologies known in the art of molecular biology (see, for example, Ausubel or Maniatis supra) in view of the teachings of the specification. For example, a vector may be constructed by inserting, into a suitable vector backbone, polynucleotides encoding a red luciferase, operably linked to a promoter of interest. Suitable vector backbones may comprise an F1 origin of replication; a colE1 plasmid-derived origin of replication; polyadenylation sequence(s); sequences encoding antibiotic resistance (e.g., ampicillin resistance) and other regulatory or control elements. Non-limiting examples of appropriate backbones include: pBluescriptSK (Stratagene, La Jolla, Calif.); pBluescriptKS (Stratagene, La Jolla, Calif.) and other commercially available vectors. Such a backbone vector may be chosen based on the cell type into which the construct is going to be introduced (e.g., bacterial cells, eucaryotic cells (e.g., plant cells, animal cells, fungal cells, insect cells, etc.)). The constructs may also contain additional reporter molecules (e.g., positive or negative selection markers).

A variety of other reporter genes may be used in the practice of the present invention. Preferred are those that produce a protein product which is easily measured in a routine assay. Suitable reporter genes include, but are not limited to chloramphenicol acetyl transferase (CAT), other light generating proteins (e.g., bioluminescent or fluorescent polypeptides), and beta-galactosidase. Convenient assays include, but are not limited to calorimetric, fluorimetric and enzymatic assays. In one aspect, reporter genes may be employed that are expressed within the cell and whose extracellular products are directly measured in the intracellular medium, or in an extract of the intracellular medium of a cultured cell line. This provides advantages over using a reporter gene whose product is secreted, since the rate and efficiency of the secretion introduces additional variables that may complicate interpretation of the assay.

Positive selection markers include any gene which a product that can be readily assayed. Examples include, but are not limited to, an HPRT gene (Littlefield, J. W., Science 145:709-710 (1964), herein incorporated by reference), a xanthine-guanine phosphoribosyltransferase (GPT) gene, or an adenosine phosphoribosyltransferase (APRT) gene (Sambrook et al., supra), a thymidine kinase gene (i.e. “TK”) and especially the TK gene of the herpes simplex virus (Giphart-Gassler, M. et al., Mutat. Res. 214:223-232 (1989) herein incorporated by reference), a nptII gene (Thomas, K. R. et al., Cell 51:503-512 (1987); Mansour, S. L. et al., Nature 336:348-352 (1988), both references herein incorporated by reference), or other genes which confer resistance to amino acid or nucleoside analogues, or antibiotics, etc., for example, gene sequences which encode enzymes such as dihydrofolate reductase (DHFR) enzyme, adenosine deaminase (ADA), asparagine synthetase (AS), hygromycin B phosphotransferase, or a CAD enzyme (carbamyl phosphate synthetase, aspartate transcarbamylase, and dihydroorotase). Addition of the appropriate substrate of the positive selection marker can be used to determine if the product of the positive selection marker is expressed, for example cells which do not express the positive selection marker nptII, are killed when exposed to the substrate G418 (Gibco BRL Life Technology, Gaithersburg, Md.).

The vector typically contains insertion sites for inserting other polynucleotide sequences of interest. These insertion sites are preferably included such that there are two sites, one site on either side of the sequences encoding the positive selection marker, luciferase and the promoter. Insertion sites are, for example, restriction endonuclease recognition sites, and can, for example, represent unique restriction sites. In this way, the vector can be digested with the appropriate enzymes and the sequences of interest ligated into the vector.

Optionally, the vector construct can contain a polynucleotide encoding a negative selection marker. Suitable negative selection markers include, but are not limited to, HSV-tk (see, e.g., Majzoub et al. (1996) New Engl. J. Med. 334:904-907 and U.S. Pat. No. 5,464,764), as well as genes encoding various toxins including the diphtheria toxin, the tetanus toxin, the cholera toxin and the pertussis toxin. A further negative selection marker gene is the hypoxanthine-guanine phosphoribosyl transferase (HPRT) gene for negative selection in 6-thioguanine.

The vectors described herein can be constructed utilizing methodologies known in the art of molecular biology (see, for example, Ausubel or Maniatis) in view of the teachings of the specification. As described above, the vector constructs containing the expression cassettes are assembled by inserting the desired components into a suitable vector backbone, for example: a vector comprising (1) a first polynucleotide having at least about 85% sequence identity to SEQ ID NO: 1, wherein said first polynucleotide encodes a polypeptide capable of mediating light-production in the presence of an appropriate substrate, e.g., luciferin, under appropriate conditions, operably linked to a transcription control element(s) of interest suitable to provide expression in a selected host cell; (2) a sequence encoding a positive selection marker; and, optionally (3) a sequence encoding a negative selection marker. In addition, the vector construct contains insertion sites such that additional sequences of interest can be readily inserted to flank the sequence encoding positive selection marker and luciferase-encoding sequence.

A preferred method of obtaining polynucleotides, suitable regulatory sequences (e.g., promoters) is PCR. General procedures for PCR as taught in MacPherson et al., PCR: A PRACTICAL APPROACH, (IRL Press at Oxford University Press, (1991)). PCR conditions for each application reaction may be empirically determined. A number of parameters influence the success of a reaction. Among these parameters are annealing temperature and time, extension time, Mg2+ and ATP concentration, pH, and the relative concentration of primers, templates and deoxyribonucleotides. After amplification, the resulting fragments can be detected by agarose gel electrophoresis followed by visualization with ethidium bromide staining and ultraviolet illumination.

In one embodiment, PCR can be used to amplify fragments from genomic libraries. Many genomic libraries are commercially available. Alternatively, libraries can be produced by any method known in the art. Preferably, the organism(s) from which the DNA is has no discernible disease or phenotypic effects. This isolated DNA may be obtained from any cell source or body fluid (e.g., ES cells, liver, kidney, blood cells, buccal cells, cerviovaginal cells, epithelial cells from urine, fetal cells, or any cells present in tissue obtained by biopsy, urine, blood, cerebrospinal fluid (CSF), and tissue exudates at the site of infection or inflammation). DNA is extracted from the cells or body fluid using known methods of cell lysis and DNA purification. The purified DNA is then introduced into a suitable expression system, for example a lambda phage. Another method for obtaining polynucleotides, for example, short, random nucleotide sequences, is by enzymatic digestion.

Polynucleotides are inserted into vector backbones using methods known in the art. For example, insert and vector DNA can be contacted, under suitable conditions, with a restriction enzyme to create complementary or blunt ends on each molecule that can pair with each other and be joined with a ligase. Alternatively, synthetic nucleic acid linkers can be ligated to the termini of a polynucleotide. These synthetic linkers can contain nucleic acid sequences that correspond to a particular restriction site in the vector DNA. Other means are known and, in view of the teachings herein, can be used.

The vector backbone may comprise components functional in more than one selected organism in order to provide a shuttle vector, for example, a bacterial origin of replication and a eucaryotic promoter. Alternately, the vector backbone may comprise an integrating vector, i.e., a vector that is used for random or site-directed integration into a target genome.

The final constructs can be used immediately (e.g., for introduction into ES cells or for liver-push assays), or stored frozen (e.g., at −20° C.) until use. In some embodiments, the constructs are linearized prior to use, for example by digestion with suitable restriction endonucleases.

The vectors are useful as reporters both in vitro and in vivo. The expression cassettes of the present invention may, for example, be introduced into a selected cell type and evaluated in culture. Further, non-invasive imaging and/or detecting of light-emitting conjugates in mammalian subjects was described in U.S. Pat. No. 5,650,135, by Contag, et al., issued Jul. 22, 1997, and herein incorporated by reference. Substrates of luciferase are typically applied to the cell or system (e.g., injection into a transgenic mouse, having cells carrying a luciferase construct, of a suitable substrate for the luciferase, for example, luciferin).

Transgenic organisms can also be produced using the sequences described herein. Constructs containing the luciferase genes are, for example, introduced into a pluripotent cell (e.g., ES cell, Robertson, E. J., In: Current Communications in Molecular Biology, Capecchi, M. R. (ed.), Cold Spring Harbor Press, Cold Spring Harbor, N.Y. (1989), pp. 39-44) by any suitable method, for example, micro-injection, calcium phosphate transformation, or electroporation (see below). After suitable ES cells containing the construct in the proper location have been identified, the cells can be inserted into an embryo, preferably a blastocyst, for example as set forth by, e.g., Bradley et al., (1992) Biotechnology, 10:534-539.

The expression cassettes of the present invention may be introduced into the genome of an animal in order to produce transgenic, non-human animals for purposes of practicing the methods of the present invention. In a preferred embodiment of the present invention, the transgenic non-human, animal may be a rodent (e.g., rodents, including, but not limited to, mice, rats, hamsters, gerbils, and guinea pigs). When a light-generating protein is used as a reporter, imaging is typically carried out using an intact, living, non-human transgenic animal, for example, a living, transgenic rodent (e.g., a mouse or rat). A variety of transformation techniques are well known in the art. Those methods include, but are not limited to, the following.

(i) Direct microinjection into nuclei: Expression cassettes can be microinjected directly into animal cell nuclei using micropipettes to mechanically transfer the recombinant DNA. This method has the advantage of not exposing the DNA to cellular compartments other than the nucleus and of yielding stable recombinants at high frequency. See, Capecchi, M., Cell 22:479-488 (1980).

For example, the expression cassettes of the present invention may be microinjected into the early male pronucleus of a zygote as early as possible after the formation of the male pronucleus membrane, and prior to its being processed by the zygote female pronucleus. Thus, microinjection according to this method should be undertaken when the male and female pronuclei are well separated and both are located close to the cell membrane. See, e.g., U.S. Pat. No. 4,873,191 to Wagner, et al. (issued Oct. 10, 1989); and Richa, J., (2001) “Production of Transgenic Mice,” Molecular Biotechnology, March 2001 vol. 17:261-8.

(ii) ES Cell Transfection: The DNA containing the expression cassettes of the present invention can also be introduced into embryonic stem (“ES”) cells. ES cell clones which undergo homologous recombination with a targeting vector are identified, and ES cell-mouse chimeras are then produced. Homozygous animals are produced by mating of hemizygous chimera animals. Procedures are described in, e.g., Koller, B. H. and Smithies, O., (1992) “Altering genes in animals by gene targeting”, Annual review of immunology 10:705-30.

(iii) Electroporation: The DNA containing the expression cassettes of the present invention can also be introduced into the animal cells by electroporation. In this technique, animal cells are electroporated in the presence of DNA containing the expression cassette. Electrical impulses of high field strength reversibly permeabilize biomembranes allowing the introduction of the DNA. The pores created during electroporation permit the uptake of macromolecules such as DNA. Procedures are described in, e.g., Potter, H., et al., Proc. Nat'l. Acad. Sci. U.S.A. 81:7161-7165 (1984); and Sambrook, ch. 16.

(iv) Calcium phosphate precipitation: The expression cassettes may also be transferred into cells by other methods of direct uptake, for example, using calcium phosphate. See, e.g., Graham, F., and A. Van der Eb, Virology 52:456-467 (1973); and Sambrook, ch.16.

(v) Liposomes: Encapsulation of DNA within artificial membrane vesicles (liposomes) followed by fusion of the liposomes with the target cell membrane can also be used to introduce DNA into animal cells. See Mannino, R. and S. Gould-Fogerite, BioTechniques, 6:682 (1988).

(vi) Viral capsids: Viruses and empty viral capsids can also be used to incorporate DNA and transfer the DNA to animal cells. For example, DNA can be incorporated into empty polyoma viral capsids and then delivered to polyoma-susceptible cells. See, e.g., Slilaty, S. and H. Aposhian, Science 220:725 (1983).

(vii) Transfection using polybrene or DEAE-dextran: These techniques are described in Sambrook, ch. 16.

(viii) Protoplast fusion: Protoplast fusion typically involves the fusion of bacterial protoplasts carrying high numbers of a plasmid of interest with cultured animal cells, usually mediated by treatment with polyethylene glycol. Rassoulzadegan, M., et al., Nature, 295:257 (1982).

(ix) Ballistic penetration: Another method of introduction of nucleic acid segments is high velocity ballistic penetration by small particles with the nucleic acid either within the matrix of small beads or particles, or on the surface, Klein, et al., Nature, 327, 70-73, 1987.

Any technique that can be used to introduce DNA into the animal cells of choice can be employed (e.g., “Transgenic Animal Technology: A Laboratory Handbook,” by Carl A. Pinkert, (Editor) First Edition, Academic Press; ISBN: 0125571658; “Manipulating the Mouse Embryo: A Laboratory Manual,” Brigid Hogan, et al., ISBN: 0879693843, Publisher: Cold Spring Harbor Laboratory Press, Pub. Date: September 1999, Second Edition.). Electroporation has the advantage of ease and has been found to be broadly applicable, but a substantial fraction of the targeted cells may be killed during electroporation. Therefore, for sensitive cells or cells which are only obtainable in small numbers, microinjection directly into nuclei may be preferable. Also, where a high efficiency of DNA incorporation is especially important, such as transformation without the use of a selectable marker (as discussed above), direct microinjection into nuclei is an advantageous method because typically 5-25% of targeted cells will have stably incorporated the microinjected DNA. Retroviral vectors are also highly efficient but in some cases they are subject to other shortcomings, as described by Ellis, J., and A. Bernstein, Molec. Cell. Biol. 9:1621 -1627 (1989). Where lower efficiency techniques are used, such as electroporation, calcium phosphate precipitation or liposome fusion, it is preferable to have a selectable marker in the expression cassette so that stable transformants can be readily selected, as discussed above.

In some situations, introduction of the heterologous DNA will itself result in a selectable phenotype, in which case the targeted cells can be screened directly for homologous recombination. For example, disrupting the gene HPRT results in resistance to 6-thioguanine. In many cases, however, the transformation will not result in such an easily selectable phenotype and, if a low efficiency transformation technique such as calcium phosphate precipitation is being used, it is preferable to include in the expression cassette a selectable marker such that the stable integration of the expression cassette in the genome will lead to a selectable phenotype. For example, if the introduced DNA contains a neo gene, then selection for integrants can be achieved by selecting cells able to grow on G418.

Transgenic animals prepared as above are useful for practicing the methods of the present invention. Operably linking a promoter of interest to a reporter sequence enables persons of skill in the art to monitor a wide variety of biological processes involving expression of the gene from which the promoter is derived. The transgenic animals of the present invention that comprise the expression cassettes of the present invention provide a means for skilled artisans to observe those processes as they occur in vivo, as well as to elucidate the mechanisms underlying those processes.

The monitoring of luciferase reporter expression cassettes using non-invasive whole animal imaging has been described (Contag, C. et al, U.S. Pat. No. 5,650,135, Jul. 22, 1997, herein incorporated by reference; Contag, P., et al, Nature Medicine 4(2): 245-247, 1998; Contag, C., et al, OSA TOPS on Biomedical Optical Spectroscopy and Diagnostics 3:220-224, 1996; Contag, C. H., et al, Photochemistry and Photobiology 66(4): 523-53 1, 1997; Contag, C. H., et al, Molecular Microbiology 18(4): 593-603, 1995). Such imaging typically uses at least one photo detector device element, for example, a charge-coupled device (CCD) camera.

Accordingly, the amount of light produced by a red luciferase encoded by a polynucleotide disclosed herein (e.g., in a cell transformed with a polynucleotide of the present invention or in a transgenic animal comprising cells expressing a red luciferase encoded by the polynucleotides of the present invention) can be quantified using either an intensified photon-counting camera or a cooled integrating camera. With respect to the cooled integrating type of camera, the particular instrument can, for example, be selected from the following three makes/models: (1) Princeton Instruments Model LN/CCD 1340-1300-EB/1; (2) Roper model LN-1300EB cooled CCD camera (available from Roper Scientific, Inc., Tucson, Ariz.); and (3) Spectral Instruments model 600 cooled CCD camera (available from Spectral Instruments, Inc., Tucson, Ariz.). A preferred apparatus is the Princeton Instruments camera number XEN-5, located at Xenogen Corporation, Alameda, California. This camera uses a charge-coupled device array (CCD array), to generate a signal proportional to the number of photons per selected unit area. The selected unit area may be as small as that detected by a single CCD pixel, or, if binning is used, that detected by any selected group of pixels. This signal may optionally be routed through an image processor, and is then transmitted to a computer (either a PC running Windows NT (Dell Computer Corporation; Microsoft Corporation, Redmond, Wash.) or a Macintosh (Apple Computer, Cupertino, Calif.) running an image-processing software application, such as “LivingImage” (Xenogen Corporation, Alameda, Calif.). The software and/or image processor are used to acquire an image, stored as a computer data file. The data generally take the form of (x, y, z) values, where x and y represent the spatial coordinates of the point or area from which the signal was collected, and z represents the amount of signal at that point or area, expressed as “Relative Light Units (RLUs).

To facilitate interpretation, the data are typically displayed as a “pseudocolor” image, where a color spectrum is used to denote the z value (amount of signal) at a particular point. Further, the pseudocolor signal image is typically superimposed over a reflected light or “photographic” image to provide a frame of reference.

It will be appreciated that if the signal is acquired on a camera that has been calibrated using a stable photo-emission standard (available from, e.g., Xenogen Corporation), the RLU signal values from any camera can be compared to the RLUs from any other camera that has been calibrated using the same photo-emission standard. Further, after calibrating the photo-emission standard for an absolute photon flux (photons emitted from a unit area in a unit of time), one of skill in the art can convert the RLU values from any such camera to photon flux values, which then allows for the estimation of the number of photons emitted per unit time, for example, by a cell transformed with a RR luciferase polynucleotide of the present invention.

The above-described cameras can be used to monitor light production mediated by the light-generating protein (e.g., a native and/or modified, optimized Red Railroad Worm red luciferase of the present invention) for both in vitro and in vivo applications.

The following examples are intended only to illustrate the present invention and should in no way be construed as limiting the subject invention.

Experimental

### EXAMPLE 1

### Modification of Phrixothrix Luciferase

Modification of a native railroad worm red luciferase-encoding sequence (GENBANK Accession No. AF139645) to a first optimized sequence (RRLUCX) was performed following the guidance of the present specification. The modified, optimized polynucleotide sequence was synthesized by Integrated DNA Technologies (Coralville, Iowa). The resulting optimized sequence did not produce light. The original native sequence was checked relative to the luciferase sequence in the clone (Ph_{RE}, described in Viviani, V. R., et al., Biochemistry 38:8271-8279, 1999) from which the original sequence was derived. The original clone (Ph_{RE}) was independently sequenced and several sequence errors were discovered relative to the AF139645 sequence. The correct sequence of the original clone is presented in the top line of FIG. 2 (SEQ ID NO: 3) and in FIG. 3 (SEQ ID NO: 3, polypeptide SEQ ID NO: 4).

The first optimized sequence RRLUCX was then modified, based on the information obtained in the independent sequence of the native isolate in order to obtain a light-generating polypeptide. Modification of the RRLUCX sequence was performed following the guidance of the present specification and using a QuikChange™ kit (Stratagene, La Jolla, Calif.) and following the manufacturer's instructions for the kit.

Table 2 (above) is a summary of the nucleic acid modifications made to the RRLUCX sequence in order to obtain the optimized, modified Red Railroad Worm luciferase sequence (labeled “RRLUCXC” in Table 2, and “RRW red LUC optimized” in FIG. 2). The nucleotide (SEQ ID NO: 1) and protein (SEQ ID NO: 2) sequences of the RRW red LUC optimized sequence are presented in FIG. 1. FIG. 2 presents a nucleotide sequence comparison between the native Red Railroad Worm luciferase (SEQ ID NO: 3) and the RRW red LUC optimized sequence (SEQ ID NO: 1).

### EXAMPLE 2

### Expression of Modified RR Luciferase in Host Cells

Plasmids expressing the modified luciferase polynucleotides are introduced into mammalian host cells to determine relative luciferase activities present in their prepared cell extracts. Plasmid DNAs are delivered into cultured mammalian cells using a modified calcium phosphate-mediated transfection procedure, as described for example in Ausubel et al. supra. Post-transfection cells are harvested and lysed. Luciferase activity of cell lysates are determined and quantified by methods known in the art, for example using the Luciferase Assay System (Promega, Madison, Wis.) and following the manufacturer's instructions. Peroxisome-modified and/or codon optimization increases expression.

### EXAMPLE 3

### In vivo Measurement of Modified Luciferases in Cells

Expression of luciferase may also be measured from living cells by adding the substrate luciferin to the growth medium. A variety of types of cells may be employed, for example, eucaryotic cells (e.g., insect, animal, mammalian, plant or fungal cells) or procaryotic cells (e.g., bacterial cells). Luminescence is thus emitted from the cells without disrupting their physiology.

In vivo expression of the luciferase reporter gene by cells can be determined, for example, by evaluating light production, mediated by the luciferase polypeptide, using a Princeton Instruments Model LN/CCD 1340-1300-EB/1 CCD camera. The cells, for example, may be grown in solution in microtiter plates and light production from each well of the microtiter plate evaluated using the CCD camera. Alternately, cells that grow on solid media may be imaged on the solid media in the presence of luciferin substrate. For example, bacteria or fungal cells expressing the modified, optimized luciferase sequence of the present invention, may be streak onto solid media plates and light production evaluated for patches and/or single colonies.

For example, bacterial cells were transformed with a plasmid having an expression cassette comprising the sequence presented as SEQ ID NO: 1. Transfected cells were selected. The transfected cells were streaked onto a plate of solid growth media. Light-output was measured from the plate using a Jobin Yvon-Spex Liquid Nitrogen Cooled Spectrophotometer (320 triple image axial direct drive system; Jobin Yvon Horiba, Edison, N.J.). The RRLUCXC polynucleotide sequence (SEQ ID NO: 1) was seen to be completely functional when expressed in the host cells and produced a light of λₘₐₓ approximately 622 nm.

As is apparent to one of skill in the art, various modification and variations of the above embodiments can be made without departing from the spirit and scope of this invention. These modifications and variations are within the scope of this invention.

What is claimed is:

1. An isolated polynucleotide, comprising a first polynucleotide having at least about 85% sequence identity to SEQ ID NO: 1, wherein said first polynucleotide encodes a polypeptide capable of mediating light-production.

2. The polynucleotide of claim 1, wherein said first polynucleotide has at least about 90% sequence identity to SEQ ID NO: 1.

3. The polynucleotide of claim 2, wherein said first polynucleotide has at least about 95% sequence identity to SEQ ID NO: 1.

4. The polynucleotide of claim 3, wherein said first polynucleotide has at least about 98% sequence identity to SEQ ID NO: 1.

5. The polynucleotide of claim 4, wherein said first polynucleotide consists of the sequence presented as SEQ ID NO: 1.

6. An expression cassette comprising the isolated polynucleotide of claim 1.

7. A cell comprising an expression cassette of claim 6.

8. A non-human, transgenic animal, comprising an expression cassette of claim 6.
